(19) United States
(12) Patent Application Publication
(10) Pub. No.: US 2013/0109079 A1
(43) Pub. Date: May 2, 2013

(54) METHOD FOR SEPARATING AN ORGANIC COMPONENT FROM A MIXTURE CONTAINING THE ORGANIC COMPONENT

(71) Applicant: Shell Oil Company, Houston, TX (US)

(72) Inventors: Christopher Andrew SPENCER, Chester (GB); Ruth ROLAND-JONES, Salisbury (GB); Wouter Spiering, Amsterdam (NL); Steven Daniel DOIG, Penzance (GB)

(73) Assignee: SHELL OIL COMPANY, Houston, TX (US)

(21) Appl. No.: 13/665,746

(22) Filed: Oct. 31, 2012

(30) Foreign Application Priority Data
Nov. 2, 2011 (EP) EP11187551.4

(51) Int. Cl. C11B 3/00; C11B 3/10; C07C 7/00; C12N 1/02

(52) U.S. Cl. 435/261; 554/175; 554/191; 585/802

(57) ABSTRACT

A method for separating an organic component from a mixture containing the organic component, water, and a microorganism, the method comprising (a) subjecting the mixture to a first separation step under mild conditions in order to generate a first stream containing water and organic component, and a second stream containing water and microorganism; and (b) subjecting the first stream to a second separation step under conditions which result in separation of the organic component from the water. The method allows for the organic component to be separated from the water under relatively severe conditions, due to the prior removal of the microorganism, and thus makes it possible to recycle the undamaged microorganism. The organic component may comprise a microbial oil which is suitable for use as, or conversion to, a biofuel.

### CROSS REFERENCE TO RELATED APPLICATIONS

This application claims the benefit of European Patent Application No. 11187551.4, filed on Nov. 2, 2011, the disclosure of which is incorporated by reference herein in its entirety.

### FIELD OF THE INVENTION

Embodiments of this invention relate to a method for separating an organic component from a mixture containing the organic component, water, and a microorganism. Embodiments of the invention also relate to a process for producing an organic component, and a process for producing a biofuel, which processes incorporate certain embodiments of the invented separation method.

### BACKGROUND

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

Hydrocarbons which may be of use as, or convertible to, fuels, can be produced using microbiological processes. For example, microorganisms can be used to catalyse the fermentation of sugars derived from plant products such as corn and sugar cane, and the fermentation products (for instance oxygenates such as fatty acids, aldehydes or alcohols) can subsequently be converted to alkanes by chemical processes such as decarboxylation, reduction and hydrogenation. In certain situations, microorganisms have been found capable of catalysing hydrocarbon syntheses by purely biochemical routes: see for example “Aliphatic and isoprenoid hydrocarbon biosynthesis for diesel fuels”, Andrew C Murley, Department of Biochemistry and Molecular Biology, Michigan State University Basic Biotech, 2009 5:1, ISSN 1944-3277.

It is also known to cultivate microorganisms such as microalgae and certain types of yeast in order to harvest the oils which they naturally secrete, or in order to extract oils from them by cell lysis. These oils from the microorganisms can be referred to as microbial oils, which can be hydrotreated to convert them into hydrocarbons, and used in the preparation of fuel components.

Other organic products of microbiological processes, for instance alcohols, can also be used as fuel components. Ethanol, for example, is widely used as a component of gasoline fuels.

Fuels which can be produced from purely natural materials, for example from plant products or by microalgae, are known as biofuels. The demand for such fuels is increasing rapidly, both in the interests of the environment and to comply with increasingly stringent regulatory demands and consumer expectations.

The product of a microbiological process for the production of an organic component such as a hydrocarbon or a microbial oil will comprise a mixture of species, including the desired organic species, the microorganism cells, water, salts, processing additives such as antifoaming additives, and often a range of organic byproducts. By way of example, a fermentation broth might contain about 0.1% to 10% w/w oil, 0.1% to 1% w/w microorganism cells and 90% to 99.8% w/w water which may itself also contain dissolved water-soluble components such as salts. At least some of the organic and aqueous components may be present in the form of an emulsion.

In order to harvest the desired organic component(s), this mixture needs to be separated into aqueous and organic phases. The separation process can however be hindered by the presence of larger organic molecules (for example C14 to C20 alkanes or fatty alcohols), which can cluster together to form small particles. These larger components need to be subjected to higher temperatures, often up to about 50° C., in order to melt them, and this can result in damage to the microorganism cells in the mixture, preventing their reuse and thus reducing the overall efficiency of the production process.

It would be desirable to provide a technique for separating organic products from such reaction mixtures, which technique could overcome or at least mitigate the above described problems.

### SUMMARY

According to one aspect of the present invention, there is provided a method for separating an organic component from a mixture containing the organic component, water, and, a microorganism, the method comprising the step of subjecting the mixture to a first separation step under a first condition to generate a first stream containing water and at least a portion of the organic component, and a second stream containing water and at least a portion of the microorganism, wherein the first condition is a mild condition. The method also comprises the step of subjecting the first stream to a second separation step under a second condition to separate at least a portion of the organic component from the water.

In one embodiment, the method further comprises the step of subjecting the second stream to a third separation step to separate at least a portion of the microorganism from the water in the second stream. In another embodiment, after the second separation step, at least a portion of the microorganism is reused in a subsequent microbiological process. In yet another embodiment, at least a portion of the microorganism being reused is active.

In certain embodiments, the mixture is a direct or indirect product of a microbiological process. In one embodiment, the mixture comprises, or is derived from, a fermentation broth. The microorganism can be selected from fungi, algae, and any combination thereof. In one embodiment, the microorganism is a non-thermophilic microorganism.

In yet another embodiment, the first separation step is carried out at a temperature of between about 30° C. and 45° C. In one embodiment, the first separation step comprises a technique selected from a gravitational separation process; a solvent extraction with a mild solvent system; a chromatography involving adsorption onto a solid substrate; a filtration process; and any combination thereof. In another embodiment, the second condition is more severe than the first condition.

In one embodiment, the organic component comprises a hydrocarbon. The hydrocarbon can comprise at least 10 carbon atoms. In that embodiment, the second condition can comprise carrying out the second separation step at a temperature sufficient to melt the hydrocarbon. In one embodiment, the temperature is greater than about 45° C.

In another embodiment, the second separation step comprises a technique selected from a solvent extraction, a distillation process, and any combination thereof. The method can also comprise a dewatering step prior to the first separation step, wherein at least a portion of the water is removed from the mixture containing the organic component. In one embodiment, the organic component comprises a microbial oil suitable for use as, and/or conversion to, a biofuel.

According to another aspect of the invention, there is provided a process for producing an organic component. The process comprises the step of subjecting the mixture to a first separation step under a first condition to generate a first stream containing water and at least a portion of the organic component, and a second stream containing water and at least a portion of the microorganism, wherein the first condition is a mild condition. The method also comprises the step of subjecting the first stream to a second separation step under a second conditions to separate at least a portion of the organic component from the water.

According to another aspect of the invention, there is provided a process for producing a biofuel, the process comprising the step of subjecting the mixture to a first separation step under a first condition to generate a first stream containing water and at least a portion of the organic component, and a second stream containing water and at least a portion of the microorganism, wherein the first condition is a mild condition. The method also comprises the step of subjecting the first stream to a second separation step under a second conditions to separate at least a portion of the organic component from the water, wherein the organic component comprises a microbial oil.

Other features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWING

The following drawing forms part of the present specification and is included to further demonstrate certain aspects of the present invention. Embodiments of the invention may be better understood by reference to the drawing in combination with the detailed description of specific embodiments presented herein.

FIG. 1 illustrates a schematic of an exemplary method, in accordance with embodiments of the invention.

### DETAILED DESCRIPTION OF PREFERRED EMBODIMENTS

By separating the organic component from the microorganism cells first, certain embodiments of the invention can make possible the use of relatively severe conditions for subsequently separating the organic component from the remaining mixture. Thus, even the larger organic species such as those requiring melting can be efficiently separated from aqueous components of the mixture. The microorganism is not subjected to these more severe (typically higher temperature) conditions; therefore, it can survive embodiments of the invention undamaged and can be reused at a subsequent time as desired or as appropriate.

Thus, embodiments of the invention can be suitable for use when the microorganism in the mixture is a microorganism other than a so-called thermophile, i.e., a microorganism which is not capable of withstanding temperatures of about 45° C. or greater. Such organisms will be referred to hereafter as non-thermophiles or as non-thermophilic microorganisms.

In one embodiment, the mixture from which the organic component is to be separated is a product of a microbiological process. A non-limiting example is a microbiological process for the production of one or more organic components, such as hydrocarbons or oils (in particular a microbial oil, e.g., oil produced by a microorganism). The mixture may be a direct or an indirect product of such a process. For example, it may have undergone one or more processing steps subsequent to the microbiological process.

In one embodiment, the microbiological process involves a microorganism-catalysed fermentation of a biological product or of a component derived from a biological product. A non-limiting example of a biological product is a plant or plant residue. A non-limiting example of a component derived from a biological product is sugar. In another embodiment, the microbiological process can involve one or more additional processing steps, particularly downstream steps, such as the hydrogenation of an alcohol or other fermentation product. Thus, in certain embodiments, the mixture containing the organic component for separation can comprise, or be derived from, a fermentation broth.

In one embodiment, the microbiological process preferably involves the production of the organic component by a microorganism such as an alga (in particular a microalga) or fungus, and optionally the cultivation of the microorganism to generate the organic product. The organic component may be secreted by such an organism, or it may be extractable from the organism. One non-limiting example of extracting the organic component is by cell lysis. In the latter case, the mixture containing the organic component for separation may already have undergone a cell lysis procedure to release the organic component from the microorganism cells.

The microorganism contained in the mixture may be selected from fungi (in particular yeasts), algae (in particular microalgae), bacteria, and any combination thereof. In particular, the microorganism may be selected from fungi, algae, and any combination thereof. It need not be, but preferably is, live. In one embodiment, the microorganism is preferably active, i.e., capable of growth and/or reproduction under appropriate conditions. As described above, it may be a non-thermophilic microorganism.

The mixture containing the organic component for separation may be obtained from a bioreactor in which an organic component-producing microorganism (for example oil-producing) has been cultivated, and/or in which a microbiological process has been carried out. Non-limiting examples of such bioreactors include fermentors and algal ponds.

In one embodiment, the organic component comprises a hydrophobic organic component, for example a lipid or oil. In a particular embodiment, it comprises a hydrocarbon component (i.e., a component containing only carbon and hydrogen atoms). Non-limiting examples of a hydrocarbon component include alkanes, alkenes, cyclic hydrocarbons, naphthenic hydrocarbons, aromatic hydrocarbons, and any combination thereof. In particular, the organic component may comprise a microbial oil that is suitable for use as, and/or conversion to, a hydrocarbon fuel. For example, such an oil may be suitable for use as, or conversion to, a biodiesel, biokerosene or biogasoline fuel, or a biocomponent of a diesel, kerosene or gasoline fuel formulation.

The organic component may comprise an oxygenate, for example selected from alcohols (including fatty alcohols); fatty acids; mono-, di- and triglycerides; lipids; ketones; aldehydes; and any combination thereof.

The organic component may comprise a mixture of two or more organic species, in particular two or more hydrocarbons, more particularly, two or more microbial oils of the type described above.

Certain embodiments of the invention may be of particular use where the organic component comprises one or more larger molecules such as those having 10 or more carbon atoms (in particular 12 or 14 or more carbon atoms, such as from 10 to 20, or from 12 to 20, or from 14 to 20 carbon atoms, as in C14 to C20 hydrocarbons), and/or one or more organic species having melting points greater than about 15° C., in particular greater than about 20° C. Certain embodiments may be of use where the mixture, from which the organic component is to be separated, comprises one or more larger organic species (for example C10 or higher, or C12 or C14 or higher), such as fatty alcohols.

In one embodiment, the organic component and the water, or at least a portion of each, may be present in the mixture in the form of an emulsion, in particular an oil-in-water emulsion. The mixture may contain other species in addition to the organic component, the water, and the microorganism. For example, the mixture may contain plant matter, process additives such as antifoam additives, salts, microorganism nutrients, and/or dissolved gases such as oxygen, nitrogen or carbon dioxide. One or more dissolved gases may be removed from the mixture before subjecting it to the invented separation method.

In one embodiment, the first stream generated in the first separation step preferably contains most of the organic component which was present in the mixture, for example about 70% or more, or about 80% or 90% or 95% or more, or about 98% or 99% or more. In another embodiment, the first stream contains all the organic component that was present in the mixture. The organic component and the water, or at least a portion of each, may be present in the first stream in the form of an emulsion, in particular an oil-in-water emulsion.

In one embodiment, the second stream generated during the first separation step preferably contains most of the microorganism that was present in the mixture, for example about 70% or more, or about 80% or 90% or 95% or more, or about 98% or 99% or more. In another embodiment, the second stream contains all the microorganism that was present in the mixture. In yet another embodiment, the second stream does not contain any of the organic component that was present in the mixture.

In a preferred embodiment, the first separation step is carried out under mild conditions. In this context, “mild conditions” or “a mild condition” refers at least to one or more conditions that are not detrimental to, or at least are not lethal to, the microorganism in the mixture. Preferably, the conditions do not permanently inactivate the microorganism, or permanently affect its ability to grow and/or reproduce.

In one embodiment, the first separation step is carried out at a temperature of less than about 45° C., preferably at a temperature of about 40° C. or less, or of about 35° C. or 30° C. or less. For example, the first separation step may be carried out at a temperature of about 15° C. or greater, or of about 20° C. or 25° C. or 30° C. or greater, or in cases of about 35° C. or 40° C. or greater, the temperature range can be between about 20° C. and 45° C. or from about 25° C. to 40° C. or from about 30° C. to 40° C. In certain cases, it may be carried out at a temperature of from about 35° C. to 44° C. or from about 40° C. to 44° C.

A range of separation techniques may be suitable for use in the first separation step. Non-limiting examples of such techniques include gravitational separation processes such as bubble or foam flotation or gravity settling; solvent extraction with a mild (suitably hydrocarbon) solvent system; chromatography involving adsorption onto a solid substrate, for example a polymer such as polypropylene, polyethylene, polystyrene, polysulphone, polyvinyl acetate or polyvinylalcohol; filtration; and any combination thereof. Again, in the context of solvent extraction, a “mild” solvent system refers at least to one that is not detrimental to, or at least is not lethal to, the microorganism in the mixture, and which suitably does not permanently inactivate the microorganism, or permanently affects its ability to grow and/or reproduce. For example, a suitable solvent system may be selected from hexane, other C6 to C12 alkanes, C6 to C12 alcohols, and any combination thereof.

The first separation step may be carried out for about 10 hours or less, or for about 7.5 or 5 hours or less, or for about 2 or 1 or 0.5 hours or less. It may be carried out for about 0.1 hours or more, or for about 0.25 or 0.5 hours or more, or for about 1 or 2 or 5 hours or more. In one embodiment, in particular when the first separation step comprises a gravitational separation process, the first separation step may be carried out for from about 1 to 10 hours, or about from 2 to 10 hours, or from about 5 to 10 hours. In another embodiment, in particular when the first separation step comprises a non-gravitational separation process, the first separation step may be carried out for from about 0.1 to 1 hours, or from about 0.1 to 0.5 hours.

The second separation step may be carried out under conditions that are more severe than those used for the first separation step. For example, the second separation step may be carried out at a higher temperature, and/or for a longer period, than the first separation step. The second separation step may involve the use of a solvent system which would be detrimental to, possibly even lethal to, the microorganism if it were present at this stage.

In one embodiment, the second separation step is carried out at a temperature of about 45° C. or higher, suitably at a temperature of about 50° C. or 75° C. or 80° C. or 85° C. or higher. It may for example be carried out at a temperature of up to about 200° C., or of up to about 175° C. or 150° C. or 140° C. or 120° C., such as from about 80° C. to 140° C.

The second separation step may be carried out for about 0.1 hours or more, or for about 0.25 or 0.5 or 0.75 or 1 hour or more. It may for example be carried out for up to about 5 hours, or for up to about 4 or 3 or 2 hours, such as for from about 0.1 to 2 hours.

A range of separation techniques may be suitable for use in the second separation step. Such techniques include solvent extraction and distillation. The solvent system used for such an extraction may be less mild than would have to be used in the first separation step, and/or the extraction may be carried out at a higher temperature and/or pressure and/or for a longer duration than would be possible in the first separation step. Non-limiting examples of a suitable solvent system for extracting the organic component during the second separation step include C4 to C24 alkanes, C3 to C12 alcohols, C6 to C10 aromatic species, chlorinated hydrocarbons, gasoline components, kerosene components, diesel components, (vacuum) gas oil, and any combination thereof.

In one embodiment, prior to the first separation step, the method can further include a dewatering step, in which at least some of the water is removed from the mixture. An advantage of such an upstream dewatering step is that a smaller volume of material needs to be processed in the subsequent first and second separation steps, leading to economies of scale which can reduce the cost and/or complexity of the overall method. The water which is removed during the dewatering step is typically free water, which is not present in emulsified form or in the form of a bound complex such as a hydrate. Preferably, the dewatering step may be used to remove up to about 25% w/w of the total water content of the mixture, or up to about 35% or 45% or 50% or 65% or 75% w/w. It may be used to remove about 5% w/w or more of the total water content of the mixture, or about 10% or 20% or 25% w/w or more, or about 30% or 40% w/w or more, for example from about 40% to 60% w/w, or approximately 50% w/w.

A range of techniques may be suitable for use in dewatering the mixture prior to the first separation step. Such techniques include filtration, centrifugation, hydrocyclonic separation, flotation, gravity settling, and combinations thereof. In one embodiment, the dewatering is preferably carried out under mild conditions, as described above in connection with the first separation step, so as not to harm the microorganism in the mixture.

In a preferred embodiment, at least a portion, often a major proportion such as about 65% w/w or more, or in cases about 70% or 80% or 90% or 95% or 99% w/w or more, of the water that remains in the mixture following the dewatering step will be present in the form of an emulsion, for example an oil-in-water emulsion containing the organic component in the discontinuous phase, or a water-in-oil emulsion containing the organic component in the continuous phase.

In certain embodiments, after the second separation step, a method according to aspects of the invention can further include one or more additional separation or processing steps. In one embodiment, the second stream which results from the first separation step may be subjected to a third separation step, in which the microorganism is separated from at least some of the water present in the stream. The third separation step may involve dewatering, for instance in the manner described above in connection with the optional pre-separation dewatering step. The third separation step may also involve a mechanical separation technique such as gravitation, centrifugation or filtration.

Water removed during the second and/or the third separation step may be cleaned and purified to an appropriate degree, and optionally recycled. For instance, it may be reused in a subsequent microbiological process, in particular a microbiological process for the production of an organic component.

In one embodiment, the microorganism is reused in a subsequent microbiological process, again in particular a microbiological process for the production of an organic component. This is possible because the microorganism has been protected from harsh separation conditions. For example, the microorganism may be recycled into the process from which it was taken prior to undergoing the method of the invention.

In one embodiment, the separated organic component may be subjected to one or more downstream processes such as hydrogenation, isomerisation, dehydration, cyclisation, esterification, etherification, desulphurization, denitrification, and any combination thereof. For example, the separated organic component may be converted into a hydrocarbon, in particular a hydrocarbon that is suitable for use as a fuel component.

According to a second aspect of the invention, embodiments for a process for producing an organic (in particular hydrocarbon) component are provided. In one embodiment, the process comprises the use of a method in accordance with the first aspect of the invention to separate the organic component from a mixture containing it. Such a process may comprise one or more upstream processing steps that result in production of a mixture containing the organic component, water and a microorganism, which mixture can then be subjected to the method of the first aspect of the invention. The one or more upstream processing steps may comprise a microbiological process, for example a fermentation process or the generation of an organic product by, or from, an organism such as a fungus or alga.

A third aspect provides embodiments for a process for producing a biofuel. In one embodiment, the process involves use of an organic component production process according to the second aspect. The organic product of the process of the second aspect of the invention may itself be suitable for use as or in a biofuel, and/or it may be subjected to one or more downstream treatment steps in order to render it suitable for use as a biofuel and/or to otherwise modify its properties. Such treatment steps may for example be selected from hydrogenation, isomerisation, dehydration, cyclisation, esterification, etherification, desulphurization, denitrification, and any combination thereof.

Throughout the description and claims of this specification, the words “comprise,” “contain,” “include,” and variations of the words, for example “comprising” and “comprises”, mean “including but not limited to”, and do not exclude other moieties, additives, components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Other features of the invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith. Moreover unless stated otherwise, any feature disclosed herein may be replaced by an alternative feature serving the same or a similar purpose.

Where upper and lower limits are quoted for a property, for example for the concentration of a fuel component, then a range of values defined by a combination of any of the upper limits with any of the lower limits may also be implied.

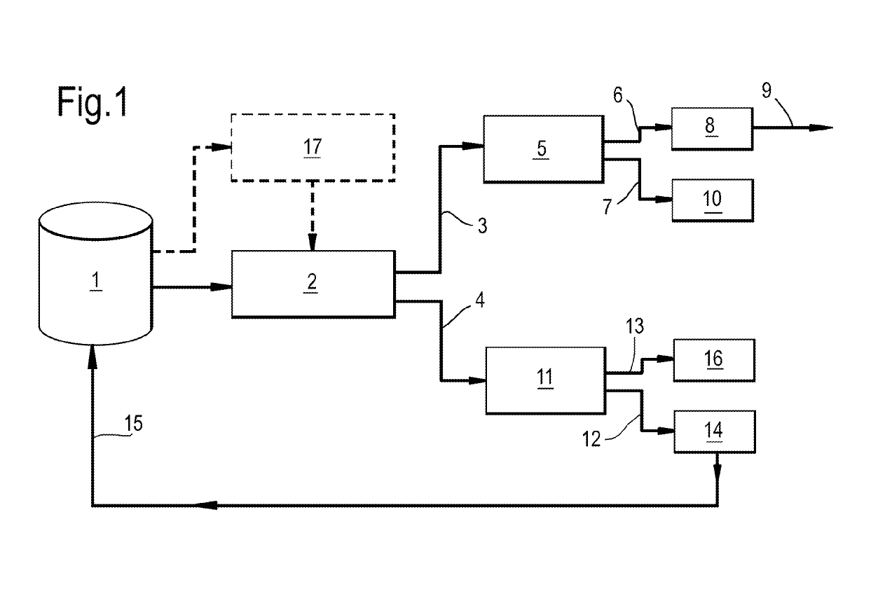

Embodiments of the present invention will now be further described with reference to the following non-limiting example and the accompanying FIG. 1, which illustrates schematically an exemplary separation method according to the invention.

### EXAMPLE

The exemplary separation method shown schematically in FIG. 1 is for use in the separation of a desired organic component—for example a microbial oil—from a mixture containing it. The mixture is produced in a bioreactor 1, preferably as a result of a microbiological process (for example, the conversion of plant biomass into oxygenates or hydrocarbons via a fermentation step, or the production of oils by microalgae cultivated in bioreactor 1). Thus, the mixture contains the desired organic component, water and microorganisms such as yeasts or algae, typically together with other reactants, additives and/or byproducts that were present at the end of the organic component production process.

The mixture containing the organic component from bioreactor 1 is fed to a first separation stage 2, at which the mixture is subjected to a first separation step under conditions that are sufficiently mild as not to damage the microorganism. This generates a first stream 3 containing the organic component and water and a second stream 4 which contains the microorganism and water. The first separation step may for instance involve filtration, and is suitably carried out at a temperature from about 30° C. to 45° C.

The first stream 3 is then fed to a second separation stage 5. Here, first stream 3 is subjected to a second separation step which separates a desired organic component 6 from the aqueous components of an output stream 7 of second separation stage 5. Because the microorganism is no longer present during the second separation step, the separation can be carried out under relatively severe conditions. It can for instance be carried out at elevated temperatures sufficient to melt larger organic species, for example from about 60° C. to 140° C.

Referring to FIG. 1, separated organic component 6 is collected at a collection stage 8. Here, separated organic component 6 may undergo appropriate and/or desired further treatment steps prior to being removed from the system as shown at 9. By way of example, organic component 6 may be a microbial oil or mixture thereof, which may be subjected to one or more treatments (such as hydrogenation, isomerisation, dehydration, cyclisation, esterification, etherification, desulphurization and/or denitrification) to render it suitable for use as a biofuel component, for instance, in an automotive gasoline or diesel fuel formulation or an aviation fuel formulation.

The separated aqueous components of stream 7 are collected at a collection stage 10. If desired, they can be recycled into bioreactor 1, or into another process, following appropriate cleaning and/or purification procedures.

Downstream of first separation stage 2, second stream 4 (which contains the microorganism and water) is fed to a third separation stage 11. Here, the microorganism is separated from at least some of the water, resulting in a microorganism-rich stream 12 and an aqueous stream 13. The microorganism-rich stream 12 is collected at stage 14. Because it has been subjected to only mild processing conditions, the microorganism is still live. It can therefore be recycled back to bioreactor 1, as shown by stream 15, for reuse. Alternatively, it can be reused in another microbiological process.

Aqueous stream 13 is directed from third separation stage 11 to a collection stage 16. Again, aqueous stream 13 can be recycled if desired, either into bioreactor 1 or into another process.

In an alternative embodiment of the invention, aqueous second stream 4 can be recycled back into bioreactor 1 without passing through third separation stage 11.

FIG. 1 also shows how the initial organic component-containing mixture may be fed from bioreactor 1, if necessary via one or more additional treatment steps, to an optional dewatering stage 17. Here, a portion (for example about 50% w/w) of non-emulsified water is removed from the mixture. This means that subsequent separation and processing steps can be carried out on smaller volumes of fluids, with consequent savings in terms of equipment capacity, time, energy input, and additional reagents such as extracting solvents and adsorption substrates, and thus also in cost.

The dewatering at 17 is preferably carried out under mild conditions so as not to harm the microorganism in the mixture. It may for instance be carried out at about 40° C. or less, using a technique such as settling. The dewatered mixture is then fed to first separation stage 2, for onward processing as described above.

It should be noted that before the start of certain embodiments of the invented method, the organic component in the mixture may have been subjected to, or be derived from, one or more treatment steps downstream of the initial microbiological process. Such steps may involve for example the conversion of a fermentation product such as an alcohol into a hydrocarbon. These treatment steps may or may not take place within bioreactor 1. They may involve the extraction of the organic component from microorganism cells, for instance by cell lysis. The mixture to be separated may be fed into first separation stage 2, or if applicable into dewatering stage 17, via such treatment steps.

It can be seen that embodiments of the present invention can provide an easily implemented yet effective way of separating a desired organic component from a mixture of organic and aqueous species which results from a microbiological process. In certain embodiments, the microorganism is isolated from the mixture under at least one mild condition, allowing the remainder of the mixture to undergo more severe separation processes such as might be necessary to remove in particular the heavier organic species. As a result, the microorganism can be reused following its separation from the organic component. Such embodiments of a process can be of particular value in the production of biofuels, including hydrocarbon biofuels.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for separating an organic component from a mixture containing the organic component, water, and a microorganism, the method comprising:
a. subjecting the mixture to a first separation step under a first condition to generate a first stream containing water and at least a portion of the organic component, and a second stream containing water and at least a portion of the microorganism, wherein said first condition is a mild condition; and
b. subjecting the first stream to a second separation step under a second condition to separate at least a portion of the organic component from the water.

2. The method of claim 1, further comprising subjecting said second stream to a third separation step to separate at least a portion of the microorganism from the water in said second stream.

3. The method of claim 1, wherein after the second separation step, at least a portion of the microorganism is reused in a subsequent microbiological process.

4. The method of claim 3, wherein at least a portion of the microorganism being reused is active.

5. The method of claim 1, wherein said mixture is a direct or indirect product of a microbiological process.

6. The method of claim 5, wherein the mixture comprises, or is derived from, a fermentation broth.

7. The method of claim 1, wherein the microorganism is selected from fungi, algae, and any combination thereof.

8. The method of claim 1, wherein the microorganism is a non-thermophilic microorganism.

9. The method of claim 1, wherein the first separation step is carried out at a temperature of between about 30° C. and 45° C.

10. The method of claim 1, wherein the first separation step comprises a technique selected from a gravitational separation process; a solvent extraction with a mild solvent system; a chromatography involving adsorption onto a solid substrate; a filtration process; and any combination thereof.

11. The method of claim 1, wherein said second condition is more severe than said first condition.

12. The method of claim 11 wherein the organic component comprises a hydrocarbon.

13. The method of claim 12 wherein the hydrocarbon comprises at least 10 carbon atoms.

14. The method of claim 13, wherein said second condition comprises carrying out the second separation step at a temperature sufficient to melt said hydrocarbon.

15. The method of claim 14 wherein said temperature is greater than about 45° C.

16. The method of claim 1, wherein the second separation step comprises a technique selected from a solvent extraction, a distillation process, and any combination thereof.

17. The method of claim 1, further comprising a dewatering step prior to the first separation step, wherein at least a portion of the water is removed from the mixture containing the organic component.

18. The method of claim 1, wherein the organic component comprises a microbial oil suitable for use as, and/or conversion to, a biofuel.

19. A process for producing an organic component, the process comprising the steps of:
a. subjecting the mixture to a first separation step under a first condition to generate a first stream containing water and at least a portion of the organic component, and a second stream containing water and at least a portion of the microorganism, wherein said first condition is a mild condition; and
b. subjecting the first stream to a second separation step under a second condition to separate at least a portion of the organic component from the water.

20. A process for producing a biofuel, the process comprising the steps of:
a. subjecting the mixture to a first separation step under a first condition to generate a first stream containing water and at least a portion of the organic component, and a second stream containing water and at least a portion of the microorganism, wherein said first condition is a mild condition; and
b. subjecting the first stream to a second separation step under a second condition to separate at least a portion of the organic component from the water, wherein the organic component comprises a microbial oil.
